(19) United States
(12) Patent Application Publication
(10) Pub. No.: US 2023/0031306 A1
(43) Pub. Date: Feb. 2, 2023

(54) INFORMATION PROCESSING DEVICE, INFORMATION PROCESSING SYSTEM, INFORMATION PROCESSING METHOD, AND INFORMATION PROCESSING PROGRAM

(71) Applicant: FUJIFILM CORPORATION, Minato-ku, Tokyo (JP)

(72) Inventors: Yasuhisa KANEKO, Kanagawa (JP); Yasuyuki HOSONO, Kanagawa (JP); Nobuya KITAMURA, Kanagawa (JP); Kenji NAGAMIYA, Kanagawa (JP); Tomohide HIRAGAMI, Kanagawa (JP)

(21) Appl. No.: 17/813,325

(22) Filed: Jul. 18, 2022

(30) Foreign Application Priority Data
Jul. 29, 2021 (JP) 2021-124694

(51) Int. Cl. G16H 50/80; G16H 50/30; H04W 4/029; H04W 4/14

(52) U.S. Cl. CPC G16H 50/80; G16H 50/30; H04W 4/029; H04W 4/14

(57) ABSTRACT

An information processing device comprising at least one processor, wherein the at least one processor is configured to transmit, in a case where a preset condition is satisfied, user information including health information about health of a user and position information of the user to a pre-registered contact. The health information may indicate at least one of a health condition or a risk that may affect health or safety for at least one of the user or another person around the user.

### CROSS-REFERENCE TO RELATED APPLICATIONS

The present application claims priority under 35 U.S.C. § 119 to Japanese Patent Application No. 2021-124694, filed on Jul. 29, 2021. The above application is hereby expressly incorporated by reference, in its entirety, into the present application.

### BACKGROUND

### Technical Field

The present disclosure relates to an information processing device, an information processing system, an information processing method, and an information processing program.

### Related Art

In the related art, a technique has been known in which another person who is close to a user is notified of a probability of infection based on position information of the user who has been specified as having been infected with an infectious disease. For example, JP2011-248802A discloses that an estimated infection date and time, which is an estimated date and time at which an infected person with a virus is infected with the virus, is acquired from a hospital and a participant who has approached the infected person within a predetermined distance after the estimated infection date and time is notified that the participant is suspected of being infected with the virus.

In order to detect various diseases, the user normally needs to take actions such as having a check-up at hospital and taking a medical examination by himself/herself. Thus, in a case where the user does not have the intention to take the action, it may be difficult to detect the disease. Especially in a case of an infectious disease, the infection may spread d by the infected person during an incubation period and an asymptomatic infected person. Therefore, a technique that can easily share information indicating that there is a probability of infection is desired. However, the technique described in JP2011-248802A requires that the infected person to be tracked be diagnosed at a hospital, which is insufficient from the viewpoint of suppressing the spread of infection.

### SUMMARY

The present disclosure provides an information processing device, an information processing method, and an information processing program capable of easily sharing health information of a user.

An information processing device according to a first aspect of the present disclosure comprises at least one processor. The processor transmits, in a case where a preset condition is satisfied, user information including health information about health of a user and position information of the user to a pre-registered contact.

In the first aspect, the health information may indicate at least one of a health condition or a risk that may affect health or safety for at least one of the user or another person around the user.

In the first aspect, the processor may estimate the health information based on text information input by at least one of the user or another person around the user via an input unit.

In the first aspect, the processor may estimate the health information based on a position where the user has been present within a preset period including a point in time at which the text information is input.

In the first aspect above, the processor may determine that the condition is satisfied in at least one of a case where biological information of the user is abnormal or a case where a result of estimating the health information indicates that there is a risk that may affect health or safety of the user.

In the first aspect, the processor may determine that the condition is satisfied in a case where the health information is acquired from another person.

In the first aspect, the processor may transmit a command regarding measurement of biological information to a transmission destination together with the user information.

In the first aspect, the command may be at least one of a command to start the measurement or a command to change a measurement condition in the measurement.

In the first aspect, the position information may indicate a non-residential location near a residential location of the user.

An information processing device according to a second aspect of the present disclosure comprises at least one processor. The processor receives user information including health information about health of another person and position information of the other person, determines whether or not the position information indicates a position within a range set in advance from at least one of a position of an own device or a preset position, and transfers the user information to a pre-registered contact in a case where the determination is affirmative.

In the second aspect, the processor may transfer the user information to the contact that belongs to a different affiliation from a user who is a transmission source of the user information.

In the second aspect, the processor may discard the received user information in a case where the same user information as the received user information has been received in the past.

In the second aspect, transfer count information indicating the number of transfers of the user information may be added to the user information, and the processor may increment the number of transfers indicated by the received transfer count information and then transfer the user information to which the transfer count information is added.

In the second aspect, the processor may not transfer the user information in a case where the number of transfers indicated by the transfer count information reaches a preset threshold value.

An information processing system according to a third aspect of the present disclosure comprises the information processing device according to the first aspect and the information processing device according to the second aspect.

In the third aspect, the transmission, reception, and transfer of the user information may be performed via at least one of e-mail or social networking service (SNS).

An information processing method according to a fourth aspect of the present disclosure executed by a computer comprises transmitting, in a case where a preset condition is satisfied, user information including health information about health of a user and position information of the user to a pre-registered contact.

An information processing method according to a fifth aspect of the present disclosure executed by a computer comprises receiving user information including health information about health of another person and position information of the other person, determining whether or not the position information indicates a position within a range set in advance from at least one of a position of an own device or a preset position, and transferring the user information to a pre-registered contact in a case where the determination is affirmative.

An information processing program according to a sixth aspect of the present disclosure causing a computer to execute a process comprises transmitting, in a case where a preset condition is satisfied, user information including health information about health of a user and position information of the user to a pre-registered contact.

An information processing program according to a seventh aspect of the present disclosure causing a computer to execute a process comprises receiving user information including health information about health of another person and position information of the other person, determining whether or not the position information indicates a position within a range set in advance from at least one of a position of an own device or a preset position, and transferring the user information to a pre-registered contact in a case where the determination is affirmative.

According to the above aspect, the information processing device, the information processing method, and the information processing program of the present disclosure can easily share health information of a user.

### BRIEF DESCRIPTION OF THE DRAWINGS

FIG. 1 is a schematic configuration diagram of an information processing system according to a first embodiment.

FIG. 2 is a block diagram showing an example of a hardware configuration of an information processing device.

FIG. 3 is a block diagram showing an example of a functional configuration of the information processing device.

FIG. 4 is a table showing an example of text information.

FIG. 5 is a table showing an example of position information.

FIG. 6 is a table showing an example of biological information.

FIG. 7 is a table showing an example of risks according to results of first determination, second determination, and third determination.

FIG. 8 is a diagram showing an example of a screen displayed on a display.

FIG. 9 is a flowchart showing an example of first information processing.

FIG. 10 is a schematic configuration diagram of the information processing system according to a second embodiment.

FIG. 11 is a table showing an example of user information.

FIG. 12 is a diagram showing an example of a screen displayed on the display.

FIG. 13 is a flowchart showing an example of second information processing.

FIG. 14 is a flowchart showing an example of third information processing.

### DETAILED DESCRIPTION

Hereinafter, embodiments for implementing a technique of the present disclosure will be described in detail with reference to drawings.

### First Embodiment

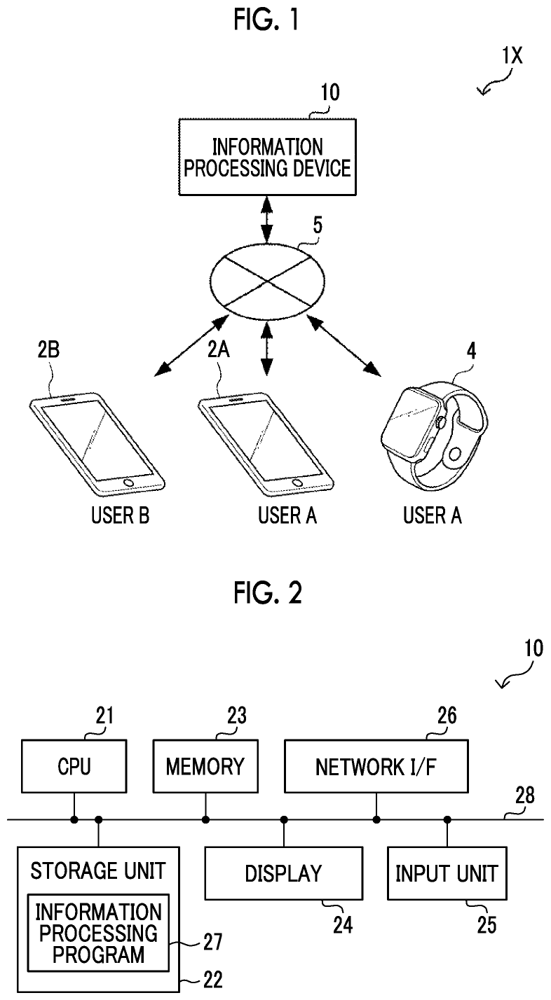

First, an example of a configuration of an information processing system 1X according to the present embodiment will be described with reference to FIG. 1. As shown in FIG. 1, the information processing system 1X comprises an information processing device 10, a plurality of terminal devices 2A and 2B, and a measurement device 4. Each of the information processing device 10, the terminal devices 2A and 2B, and the measurement device 4 can communicate with each other via a wired or wireless network 5. The plurality of terminal devices 2A and 2B are owned by different users. Hereinafter, it is assumed that a user A owns the terminal device 2A and the measurement device 4, and a user B owns the terminal device 2B, as shown in FIG. 1. In a case where the terminal devices 2A and 2B are not distinguished in the description, the terminal devices are simply referred to as “terminal device 2”.

The terminal device 2 has a function that a user who is an owner can input text information by an input unit such as a touch panel and a keyboard. The terminal device 2 further has a function of positioning a position of an own device and recording position information by using, for example, a global positioning system (GPS). As the terminal device 2, for example, portable devices such as a laptop, a tablet terminal, and a wearable terminal can be applied as appropriate, in addition to a smartphone shown in FIG. 1.

The measurement device 4 has a function of measuring at least one type of biological information of the user. The biological information indicates, for example, at least one of body temperature, heartbeat, electrocardiogram, myoelectricity, blood pressure, arterial oxygen saturation (SpO2), blood glucose level, lipid level, or the like. In these cases, for example, a wearable device such as a smartwatch provided with a thermometer, a heart rate monitor, a blood glucose self-measurement device, a sensor that measures the biological information such as heartbeat and arterial oxygen saturation, and the like can be applied as the measurement device 4. In a case where there is a command to measure the biological information, the measurement device 4 may measure the biological information only once or may measure the biological information over time. The term “measurement over time” means to continuously measure the biological information at preset time intervals.

The measurement device 4 may further have a function of transmitting the measured biological information to the information processing device 10 and/or the terminal device 2A by wired or wireless communication. As the wireless communication standard in this case, for example, Wi-Fi (registered trademark), Bluetooth (registered trademark), and the like can be applied as appropriate. Although FIG. 1 shows one measurement device 4, the number of measurement devices 4 included in the information processing system 1X according to the present embodiment is not particularly limited. For example, in a case where the information processing system 1X includes a plurality of measurement devices 4, each of the plurality of measurement devices 4 may be enabled to communicate with the information processing device 10 and/or the terminal device 2A by wired or wireless communication. Further, each of the plurality of measurement devices 4 may measure different types of the biological information.

The information processing device 10 according to the present embodiment estimates a risk that may affect health or safety of the user according to the text information input by the user, the position information of the user (that is, action of the user), and the biological information of the user to specify a user having the risk at an early stage. Hereinafter, a detailed configuration of the information processing device 10 will be described.

Next, an example of a hardware configuration of the information processing device 10 according to the present embodiment will be described with reference to FIG. 2. As shown in FIG. 2, the information processing device 10 includes a central processing unit (CPU) 21, a nonvolatile storage unit 22, and a memory 23 as a temporary storage area. The information processing device 10 further includes a display 24 such as a liquid crystal display, an input unit 25 such as a keyboard, a mouse, a touch panel, or a button, and a network interface (I/F) 26. The network I/F 26 is connected to the network 5 and performs wired or wireless communication. The CPU 21, the storage unit 22, the memory 23, the display 24, the input unit 25, and the network I/F 26 are mutually connected, via a bus 28 such as a system bus and a control bus, such that various pieces of information can be exchanged.

The storage unit 22 is formed by a hard disk drive (HDD), a solid state drive (SSD), a flash memory, and the like. The storage unit 22 stores an information processing program 27 in the information processing device 10. The CPU 21 reads out the information processing program 27 from the storage unit 22, develops the readout program into the memory 23, and executes the developed information processing program 27. The CPU 21 is an example of a processor of the present disclosure. As the information processing device 10, for example, a personal computer, a server computer, a smartphone, a tablet terminal, a wearable terminal, or the like can be applied as appropriate.

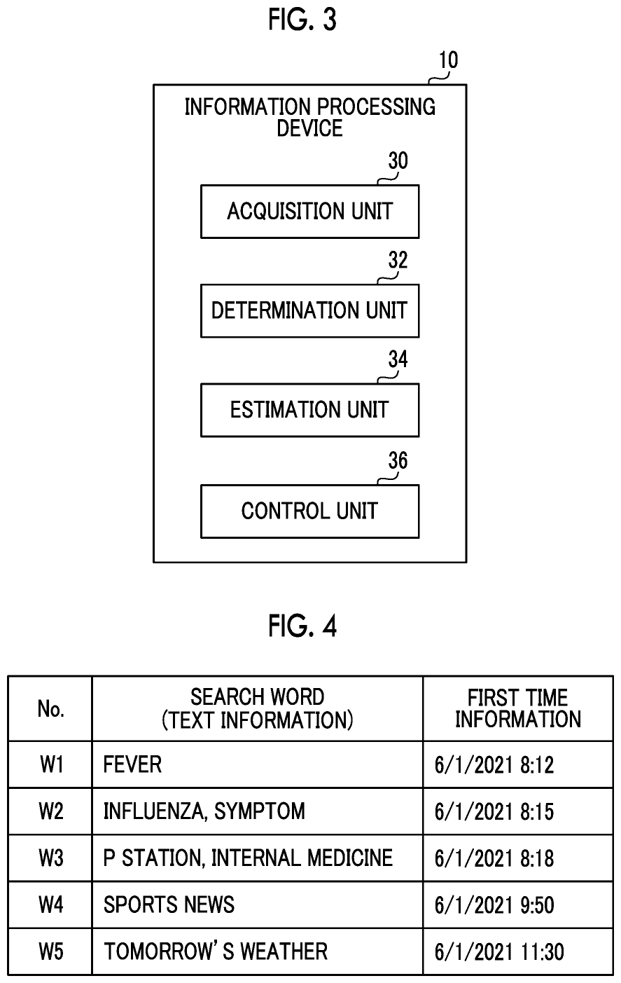

Next, an example of a functional configuration of the information processing device 10 according to the present embodiment will be described with reference to FIG. 3. As shown in FIG. 3, the information processing device 10 includes an acquisition unit 30, a determination unit 32, an estimation unit 34, and a control unit 36. The CPU 21 executes the information processing program 27 to function the CPU 21 as the acquisition unit 30, the determination unit 32, the estimation unit 34, and the control unit 36.

In the following description, it is assumed that the information processing device 10 acquires various pieces of information from the terminal device 2A and the measurement device 4 owned by the user A in FIG. 1 and estimates the risk that may affect the health or safety of the user A. It is assumed that the owner of the terminal device 2A and the measurement device 4 can be distinguished by storing the terminal device 2A, the measurement device 4, and the user in the storage unit 22 in advance in association with each other.

The acquisition unit 30 acquires the text information to which first time information indicating an input point in time is added from the terminal device 2A via the network 5 by the network I/F 26. The text information is, for example, a character string input by the user A using the input unit of the terminal device 2A and may be a search word or the like input to a search engine. FIG. 4 shows a search word as an example of the text information to which the first time information (that is, information indicating a searched point in time) is added. FIG. 4 shows five search words in a time series. In a case where these search words are to be distinguished below, these search words are referred to, using reference numerals described in a field of “No.”, as “search word W1” to “search word W5”. In the present specification, the term “point in time” means one time point represented by a year, a month, a day, an hour, a minute, and a second.

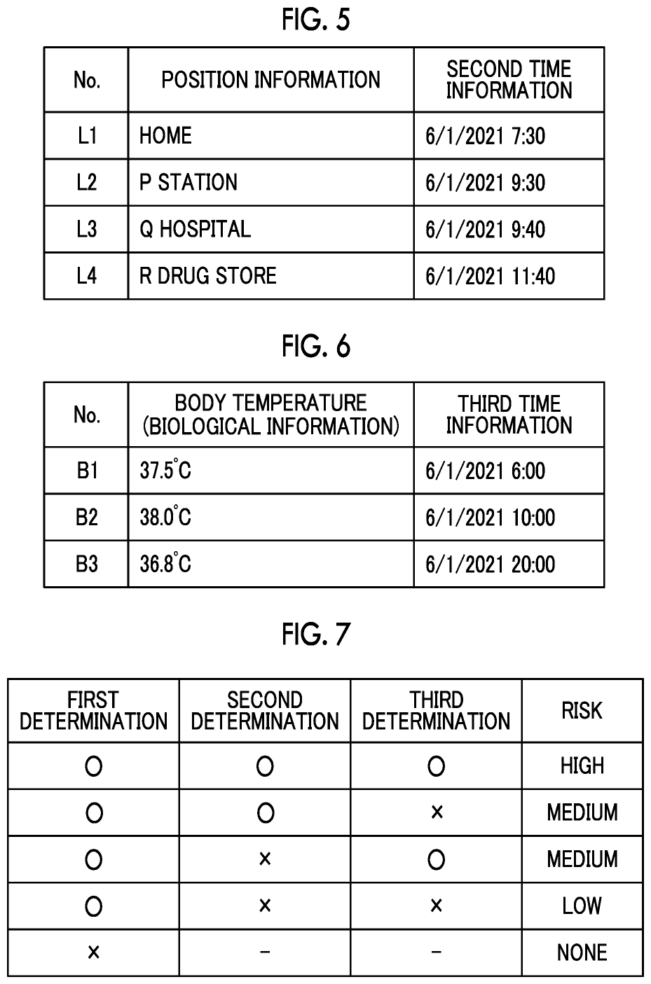

The acquisition unit 30 acquires, from the terminal device 2A via the network 5 by the network I/F 26, the position information of the user A and to which second time information indicating a point in time at which the user A has been present at a position indicated by the position information is added. The “position information of the user A” is, that is, the position information of the terminal device 2A owned by the user A. FIG. 5 shows an example of the position information to which the second time information is added. FIG. 5 shows four pieces of position information in a time series. In a case where these pieces of position information are to be distinguished below, these pieces of position information are referred to, using reference numerals described in a field of “No.”, as “position information L1” to “position information L4”. Although a facility name and the like are shown as the position information in FIG. 5 for the sake of clarity, the position information may be represented by latitude and longitude, address notation, and the like.

The acquisition unit 30 acquires, from the measurement device 4 via the network 5 by the network I/F 26, the biological information measured from the user A and to which third time information indicating a measurement point in time is added. FIG. 6 shows the body temperature as an example of the biological information to which the third time information is added. FIG. 6 shows three body temperatures in a time series. In a case where these body temperatures are to be distinguished below, these body temperatures are referred to, using reference numerals described in a field of “No.”, as “body temperature B1” to “body temperature B3”.

First, the determination unit 32 makes first determination as to whether or not the text information acquired by the acquisition unit 30 includes a preset keyword. The “preset keyword” is, for example, related to a health condition of the user A. Examples of such a keyword include keywords related to symptoms such as “fever”, “headache”, “cough”, and “symptom”, keywords related to disease names such as “cold”, and keywords related to check-ups at hospital such as “hospital” and “internal medicine”. Among the keywords, the “preset keyword” may indicate a probability of infection of the user A with an infectious disease. Examples of such a keyword include keywords related to infectious diseases such as “influenza virus”, “coronavirus” and “food poisoning”. The preset keyword is stored in, for example, the storage unit 22.

In the example of FIG. 4, the determination unit 32 determines that the search words W1, W2, and W3 include the preset keyword. Since it can be said that the search word reflects subjective symptoms related to the health condition of the user A, it is presumed that the user A feels abnormality in the user's health condition from the search words W1, W2, and W3 and considers the check-up at hospital.

Secondly, based on the position information to which the second time information indicating a point in time within a preset first period that includes the point in time indicated by the first time information, added to the text information determined to include the preset keyword in the first determination, is added among the position information acquired by the acquisition unit 30, the determination unit 32 makes second determination as to whether or not the user A has been present at a preset position within the first period. That is, in the second determination, determination is made whether or not the user A has gone to the preset position within the preset first period from a time point at which the user A inputs the preset keyword. The “preset position” is, for example, a location of a medical institution such as a hospital or a pharmacy. As the “first period”, an appropriate period can be randomly set as a period from the user's feeling of the abnormality in the user's health condition until the user goes to the medical institution and may be, for example, several hours to several days.

Based on the position information to which the second time information indicating the point in time after the point in time indicated by the first time information, added to the text information determined to include the preset keyword in the first determination, is added, the determination unit 32 may make determination, as the second determination, as to whether or not the user A has been present at the preset position. That is, in the second determination, the user A may input the preset keyword, and then determination may be made whether or not the user A has gone to the preset position. This is because, in general, the user often goes to the medical institution after the user feels the abnormality in the user's health condition, and the action before the user feels the abnormality in the user's health condition may be considered to be ignored.

The determination unit 32 may change the first period according to the keyword included in the text information. For example, the first period may be changed according to an average recovery period of the disease indicated by the keyword, an incubation period of the infectious disease, and the like. A correspondence relationship between the keyword and the first period is stored in, for example, the storage unit 22.

The determination unit 32 may determine, as the second determination, whether or not the user A has been present at the preset position for the preset second period or longer based on the position information. As the “second period”, an appropriate period can be randomly set as a period for the user to stay at the medical institution and may be, for example, several tens of minutes to several hours. The second period is shorter than the first period. With such a configuration, for example, a case where the user A only passes through the medical institution can be excluded.

As an example, it is assumed that the first period is set to be within 3 hours from the point in time indicated by the first time information, added to the text information determined to include the preset keyword in the first determination, and the second period is set to 30 minutes. In the examples of FIGS. 4 and 5, based on the position information L3 to which the second time information indicating the point in time within the first period (8:18 to 11:18 on Jun. 1, 2021) indicating within 3 hours from the point in time indicated by the first time information of the search word W3 is added, the determination unit 32 determines that the user A has been present in a “Q hospital” within the first period. The determination unit 32 determines that the user A has been present in the “Q hospital” for 30 minutes or more based on a time interval between the second time information of the position information L3 and the second time information of the position information L4. According to this second determination, it is presumed that the user A feels the abnormality in the user's health condition and then actually has the check-up at hospital.

Thirdly, based on the biological information to which the third time information indicating a point in time within a preset third period that includes the point in time indicated by the first time information, added to the text information determined to include the preset keyword in the first determination, is added among the biological information acquired by the acquisition unit 30, the determination unit 32 makes third determination as to whether or not the biological information indicates the abnormality. That is, in the third determination, determination is made whether or not the biological information indicates the abnormality within the preset third period from the time point at which the user A inputs the preset keyword. The determination unit 32 may compare the biological information with a preset threshold value to make the third determination as to whether or not the biological information indicates the abnormality. As the “third period”, an appropriate period can be randomly set as a time interval between a time point at which the user feels the abnormality in the user's health condition and a time point at which the abnormality occurs in the biological information and may be, for example, several hours to several days.

As an example, it is assumed that the threshold value for abnormal body temperature is set to “37.0° C.” and the third period is 3 hours before and after the point in time indicated by the first time information, added to the text information determined to include the preset keyword in the first determination. In the examples of FIGS. 4 and 6, based on the body temperatures B1 and B2 to which the third time information indicating the point in time within the third period (“5:18 to 11:18 on Jun. 1, 2021”) indicating within 3 hours before and after the point in time indicated by the first time information of the search word W3 is added, the determination unit 32 determines that the body temperature indicates the abnormality. According to this third determination, it can be seen that in a case where the user A feels the abnormality in the user's health condition, the biological information actually indicates the abnormality.

The estimation unit 34 estimates the risk that may affect the health or safety of the user (hereinafter, simply referred to as “risk”) according to results of the first determination, the second determination, and the third determination by the determination unit 32. FIG. 7 shows an example of a table in which risks corresponding to combinations of the results of the first determination, the second determination, and the third determination are preset. In fields of first determination, second determination, and third determination, a circle mark means that each piece of determination is affirmative determination, and a cross mark means that each piece of determination is negative determination. In a field of risk, the risk is evaluated as “high”, “medium”, “low”, and “none” in descending order. By collating with the table of FIG. 7, the estimation unit 34 estimates the presence or absence and magnitude of the risk according to the results of the first determination, the second determination, and the third determination by the determination unit 32.

As described above, all of the results of the first determination, the second determination, and the third determination in the examples of FIGS. 4 to 6 are the affirmative determination. Thus, the estimation unit 34 estimates the risk in the examples of FIGS. 4 to 6 as “high” based on the table of FIG. 7.

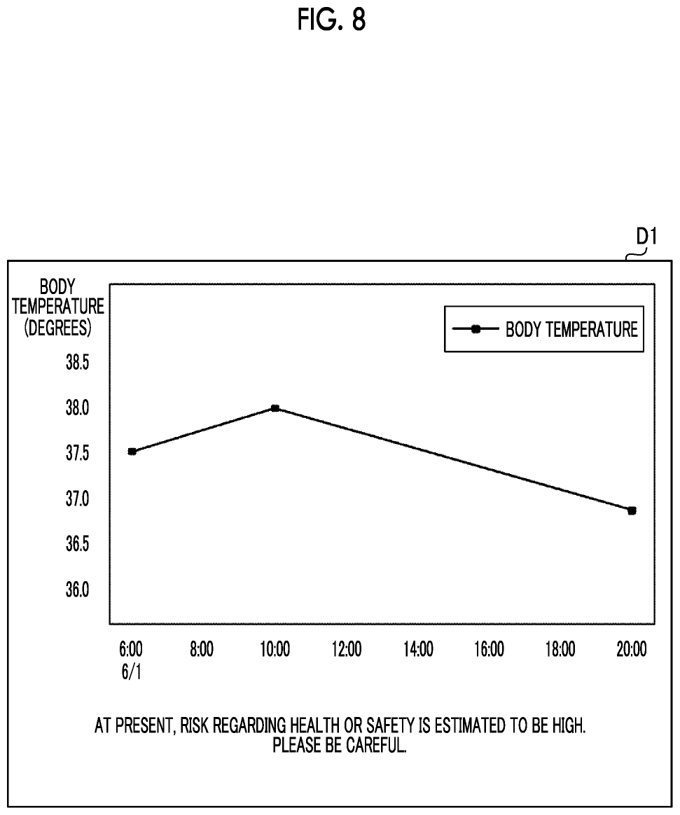

The control unit 36 notifies the terminal device 2A of the risk estimated by the estimation unit 34. The control unit 36 may further control to display the risk estimated by the estimation unit 34 on a display of the terminal device 2A. FIG. 8 shows an example of a screen D1 displayed on the display of the terminal device 2A. By viewing the screen D1, the user A can check that there is the risk that may affect the health or safety of the user.

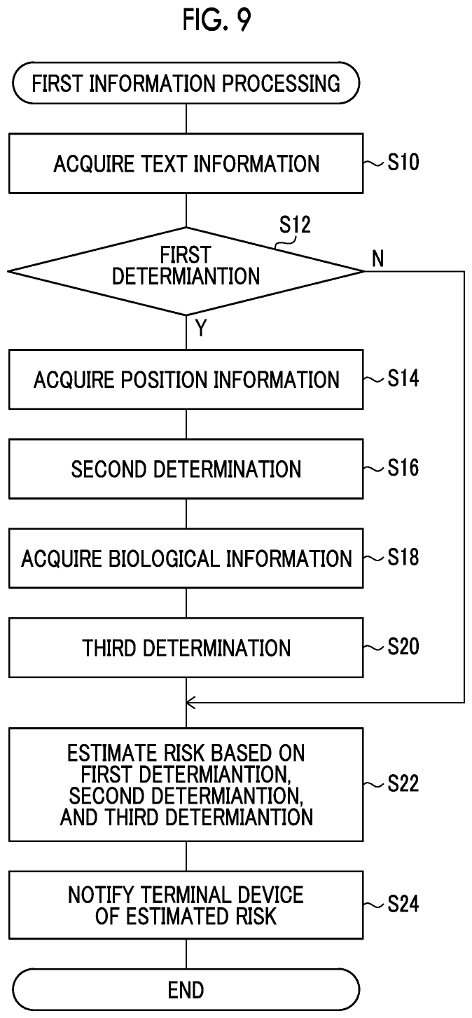

Next, an action of the information processing device 10 according to the present embodiment will be described with reference to FIG. 9. In the information processing device 10, the CPU 21 executes the information processing program 27 to execute first information processing shown in FIG. 9. The first information processing is executed, for example, in a case where the user gives an instruction to start execution via the input unit 25.

In step S10, the acquisition unit 30 acquires, from the terminal device 2, text information to which the first time information indicating the input point in time is added. In step S12, the determination unit 32 makes the first determination as to whether or not the text information acquired in step S10 includes the preset keyword. In a case where the affirmative determination is made in step S12 (that is, in a case where the text information includes the preset keyword), the processing proceeds to step S14.

In step S14, the acquisition unit 30 acquires, from the terminal device 2, the position information to which the second time information indicating the point in time at which the user has been present at the position indicated by the position information is added. In step S16, based on the position information to which the second time information indicating a point in time within a preset first period that includes the point in time indicated by the first time information, added to the text information determined to include the preset keyword in the first determination in step S12, is added among the position information acquired in step S14, the determination unit 32 makes the second determination as to whether or not the user has been present at the preset position within the first period.

In step S18, the acquisition unit 30 acquires, from the measurement device 4, the biological information to which the third time information indicating the measurement point in time is added. In step S20, based on the biological information to which the third time information indicating a point in time within a preset third period that includes the point in time indicated by the first time information, added to the text information determined to include the preset keyword in the first determination in step S12, is added among the biological information acquired in step S18, the determination unit 32 makes the third determination as to whether or not the biological information indicates the abnormality.

In step S22, the estimation unit 34 estimates the risk that may affect the health or safety of the user based on the result of the first determination in step S12, the result of the second determination in step S16, and the result of the third determination in step S20. In step S24, the control unit 36 controls to notify the terminal device 2 of the risk estimated in step S22 and ends the first information processing.

In a case where the negative determination is made in step S12 (that is, the text information does not include the preset keyword), steps S14 to S20 may be omitted and the processing may proceed to step S22. In this case, in step S22, the estimation unit 34 may estimate that there is no risk based on the result of the first determination in step S12. In the first information processing, the order of each step may be changed as appropriate, such as performing the processing of steps S14 and S18 before step S12.

As described above, the information processing device 10 according to one aspect of the present disclosure comprises at least one processor. The processor acquires the text information to which the first time information indicating the input point in time is added and the position information of the user to which the second time information indicating the point in time at which the user has been present at the position indicated by the position information is added. The first determination is made as to whether or not the text information includes the preset keyword. In addition, based on the position information to which the second time information indicating the point in time within the preset first period that includes the point in time indicated by the first time information, added to the text information determined to include the preset keyword in the first determination, is added, the second determination is made as to whether or not the user has been present at the preset position. The risk that may affect the health or safety of the user is estimated according to the results of the first determination and the second determination.

With such an information processing device 10, the risk that may affect the health or safety of the user can be estimated based on the text information that the user inputs on a daily basis and the daily action of the user. Therefore, it is possible to check the health condition of the user without being aware of the user. Therefore, it is possible to specify the user having the risk at an early stage.

In the first embodiment, the determination unit 32 makes the first determination, the second determination, and the third determination, and the estimation unit 34 estimates the risk according to the results of the first determination, the second determination, and the third determination. However, the present disclosure is not limited thereto. In the information processing device 10 according to the first embodiment, the third determination may be omitted. That is, the determination unit 32 may make the first determination and the second determination, and the estimation unit 34 may estimate the risk that may affect the health or safety of the user according to the results of the first determination and the second determination.

In the first embodiment, as an example of the text information, the search word input by the user who is the owner of the terminal device 2 is used. However, the present disclosure is not limited thereto. The acquisition unit 30 can acquire information addressed to another person by the user and information addressed to the user by another person, as the text information. For example, a message input by the user oneself or another person in at least one of e-mail or a social networking service (SNS) may be applied as the text information. The e-mail is not limited to e-mail using a protocol such as simple mail transfer protocol (SMTP) and includes, for example, various services having a function of transmitting and receiving messages such as webmail and short message service (SMS). The SNS includes various services having a function of transmitting and receiving messages such as Twitter (registered trademark), Facebook (registered trademark), and LINE (registered trademark).

For example, a doctor's diagnosis result, a health examination result, and the like related to the health condition of the user, which are transmitted from a server computer or the like that manages an electronic medical record to the terminal device 2, may be applied as the text information. In a case where the acquisition unit 30 acquires these pieces of information, the determination unit 32 determines, as the first determination, that the keyword related to the health condition of the user is included.

For example, a notification indicating that the user who is the owner of the terminal device 2 may have contacted a person infected with the coronavirus disease, which is notified to the terminal device 2 using a known coronavirus contact check application, may be applied, as the text information. In a case where the acquisition unit 30 acquires this notification, the determination unit 32 determines, as the first determination, that the keyword indicating the probability of infection of the user with the infectious disease is included.

In the first embodiment, the keyword used in the first determination is related to the health condition of the user A. However, the present disclosure is not limited thereto. For example, the keyword used in the first determination may be related to the health condition of another person around the user. Among the keywords, the keyword may indicate an infection state of another person around the user with the infectious disease. This is because in a case where another person around the user is infected with the infectious disease, the fact may affect the health condition of the user. Examples of such a keyword include the keywords indicating the health condition of another person around the user such as “fever”, “went to hospital”, and “infected with influenza”, which are included in the message addressed to the user by another person.

For example, the keyword used in the first determination may be related to a disaster or pollution around the user. For example, a notification indicating that a disaster, such as fire and gas leak, or pollution has occurred in an area where the user who is the owner of the terminal device 2 stays may be applied, as the text information. This is because in a case where the disaster or pollution has occurred around the user, the fact may affect the health condition and safety of the user. In a case where the acquisition unit 30 acquires this notification, the determination unit 32 determines, as the first determination, that the keyword related to the disaster or pollution around the user is included.

In the first embodiment, the risk is estimated based on the text information, position information, and biological information of the user. However, the present disclosure is not limited thereto. For example, in a case where the user is infected with the infectious disease, it is regarded that there may be the same risk as the user also for another person around the user.

Specifically, the acquisition unit 30 acquires the position information about each of a plurality of different users. For a second user whose position information indicates that the user has been present at the same position in the same time zone as a first user estimated to have the risk in the risk estimation, the estimation unit 34 estimates that the second user has the same risk as the first user. The control unit 36 notifies the terminal device 2 owned by the second user of the risk estimated by the estimation unit 34. In the “same time zone”, each point in time indicated by the second time information added to the position information about each user may be included within a preset period. In the “same position”, a distance between positions indicated by the position information about each user may be within a preset distance.

For example, in the example of FIG. 1, the acquisition unit 30 acquires the position information from each of the terminal device 2A owned by the user A and the terminal device 2B owned by the user B. It is assumed that each piece of position information indicates that the user A and the user B have been present at the same position in the same time zone. In a case where the estimation unit 34 estimates that there is the risk related to the user A, the estimation unit 34 estimates that the user B who has been present at the same position in the same time zone as the user A has the same risk as the user A.

In the first embodiment, the location of the medical institution such as the hospital and the pharmacy are exemplified as the “preset position” used for the second determination. However, the present disclosure is not limited thereto. The “preset position” used for the second determination may be a location of a restaurant. In general, it is said that the spread of the infectious disease is likely to occur in restaurants. By notifying a user who has gone to the restaurant among users who have input the preset keyword (that is, users having a subjective symptom) that the risk is high, a warning can be issued to the user. As described above, by notifying another person whose position information indicates that the person has been present at the same restaurant in the same time zone as the user estimated to have the risk that there is the same risk as the user subjected to the risk estimation, another person is urged to be cautious.

In the information processing system 1X according to the first embodiment, at least a part of the information processing device 10, the terminal device 2, and the measurement device 4 may be the same device. For example, the terminal device 2 may have some or all of the functions of the acquisition unit 30, the determination unit 32, the estimation unit 34, and the control unit 36 related to the information processing device 10. In this case, the acquisition unit 30 may acquire the text information input in the own device and the position information positioned in the own device. For example, the terminal device 2 may have the function of measuring the biological information, and the acquisition unit 30, instead of the measurement device 4, may acquire the biological information measured by the own device.

### Second Embodiment

In the first embodiment, in a case where it is estimated that there is the risk that may affect the health or safety of the user, the terminal device 2 owned by the user is notified of the fact. By the way, in order to prevent the spread of infectious diseases, it may be effective to share information about the health condition and risk of the user promptly by transmitting the information from the user to another person around the user and further transferring the information from the other person to the other person around the other person.

The information processing device according to a second embodiment has a function of transmitting or transferring the information about the health condition and risk of the user to an information processing device owned by another person around the user. Hereinafter, an information processing system 1Y and functions of the information processing device 10 according to the second embodiment will be described, but the same functions and configurations as those of the first embodiment will be omitted.

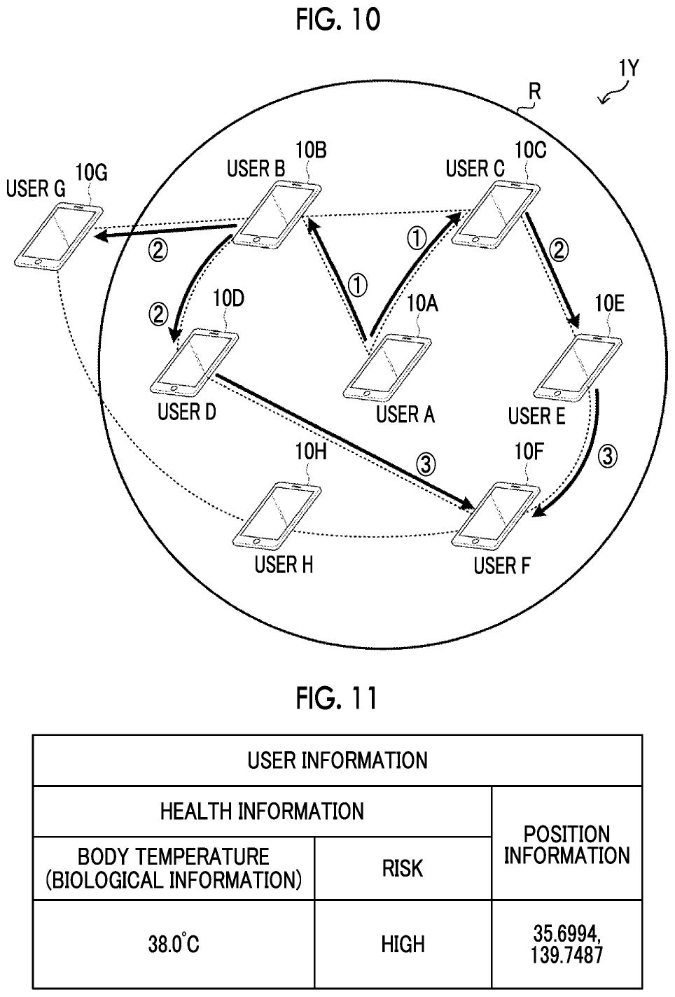

First, an example of a configuration of the information processing system 1Y according to the present embodiment will be described with reference to FIG. 10. As shown in FIG. 10, the information processing system 1Y comprises information processing devices 10A to 10H owned by a plurality of users A to H, respectively. The information processing devices 10A to 10H can communicate with each other via a wired or wireless network (not shown), respectively. Each of the information processing devices 10A to 10H is assumed to comprise the functions and configuration of the terminal device 2 in addition to the functions and configuration of the information processing device 10 in the first embodiment (in other words, the terminal device 2 in the first embodiment comprises the functions and configuration of the information processing device 10). Hereinafter, in a case where the information processing devices 10A to 10H are not distinguished, the devices are simply referred to as “information processing device 10”. In a case where users A to H are not distinguished, the users are simply referred to as “user”.

In the following description, in the information processing system 1Y, it is assumed that the information processing device 10A of the user A serves as a transmission source of user information (details will be described below) about the user A, and the information processing devices 10B to 10H transfers the user information about the user A according to a determination result shown below. Illustrated positions of the information processing devices 10A to 10H in FIG. 10 represent presence positions of the users A to H. For example, the users B to G represent that the users exist within a certain range R from the presence position of user A, and the user H represents that the user exists outside a certain range R from the presence position of user A. A solid arrow shown in FIG. 10 indicates a transmission route or a transfer route of the user information, and a circled number means the number of transfers (details will be described below). A dotted line shown in FIG. 10 means that the information processing devices 10 connected by the dotted line are pre-registered contacts (details will be described below).

Next, an example of a functional configuration of the information processing device 10A owned by the user A, which is the transmission source of the user information, will be described. As shown in FIG. 3, similarly to the information processing device 10 according to the first embodiment, the information processing device 10A includes the acquisition unit 30, the determination unit 32, the estimation unit 34, and the control unit 36. The CPU 21 executes the information processing program 27 to function the CPU 21 as the acquisition unit 30, the determination unit 32, the estimation unit 34, and the control unit 36.

The acquisition unit 30 acquires the text information input by the user A, the position information of the user A, and the biological information of the user A in the same manner as in the first embodiment. The determination unit 32 makes the first determination based on the text information, the second determination based on the position information, and the third determination based on the biological information in the same manner as in the first embodiment. The estimation unit 34 estimates the risk that may affect the health or safety of the user A based on the results of the first determination, the second determination, and the third determination by the determination unit 32 in the same manner as in the first embodiment.

The estimation unit 34 generates health information about the health of the user A. The “health information” indicates at least one of the health condition of the user A or the risk, which is estimated by the estimation unit 34, that may affect the health or safety of the user A (refer to FIG. 11). That is, the estimation unit 34 may estimate and generate the health information (specifically, the risk) of the user A based on the position where the user A has been present within the preset first period including the point in time at which the text information acquired by the acquisition unit 30 is input.

The “health condition” is indicated by, for example, the biological information acquired by the acquisition unit 30. The “biological information” may be, for example, at least one of body temperature, heartbeat, electrocardiogram, myoelectricity, blood pressure, SpO2, blood glucose level, lipid level, or the like measured by the measurement device 4. For example, the doctor's diagnosis result and the health examination result related to the health condition of the user A, an examination result related to various diseases such as infectious diseases, and the like, which are transmitted from the server computer or the like that manages the electronic medical record to the information processing device 10A, may be applied as the “biological information”.

For example, the estimation unit 34 may estimate and generate the health information of the user A based on the text information input by at least one of the user A and another person around the user A via the input unit 25. Specifically, in a case where the text information includes a keyword related to the health condition of the user A, the estimation unit 34 may estimate the health condition of the user A according to the keyword and generate the estimated result as the health information. For example, in a case where the text information includes the keyword “influenza virus”, the estimation unit 34 estimates that the user A may be infected with an influenza virus infectious disease and generates the health information indicating that fact.

As the keyword used by the estimation unit 34 for estimating the health condition, the same keyword as that used in the first determination by the determination unit 32 described in the first embodiment can be applied. That is, the keyword related to the health condition of the user A, the keyword indicating the probability of infection of the user A with the infectious disease, the keyword related to the health condition of another person around the user A, the keyword indicating the infection state of another person around the user A with the infectious disease, and the like can be applied.

In a case where a preset condition is satisfied, the control unit 36 controls to transmit, to the pre-registered contact, the user information including the health information about the health of the user A generated by the estimation unit 34 and the position information of the user A acquired by the acquisition unit 30. For example, in a case where the result of estimating the health information by the estimation unit 34 indicates that there is the risk that may affect the health or safety of the user A, the control unit 36 may determine that the condition is satisfied. In this case, whether or not the condition is satisfied may be changed according to the magnitude of the estimated risk such as determining that the condition is satisfied in a case where the risk is estimated to be “high” and determining that the condition is not satisfied in a case where the risk is estimated to be “medium” and “low”. For example, in a case where the biological information of the user A is determined to be abnormal, the control unit 36 may determine that the condition is satisfied. In this case, the control unit 36 can determine whether or not the biological information indicates the abnormality by comparing the biological information with the preset threshold value.

FIG. 11 shows an example of the user information. The user information shown in FIG. 11 includes the biological information (body temperature) of the user A, the health information indicating the risk of the user A, and the position information of the user A. In FIG. 11, the position information is represented by latitude and longitude.

The transmission of the user information can be performed, for example, via at least one of e-mail or SNS. The “pre-registered contact” is a contact whose e-mail address is stored in advance in an external storage medium, such as the storage unit 22 or a cloud server, in a case where the transmission unit is e-mail. In a case where the transmission unit is SNS, the “pre-registered contact” is a contact to which the account is associated such that various pieces of information can be transmitted and received to each other in advance.

The example of FIG. 10 shows that the information processing devices 10B and 10C are registered as the contacts in advance in the information processing device 10A. It is mutually known in advance that the information processing devices 10A to 10C belong to the same group by functions such as a known group talk in SNS and a mailing list using e-mail. On the other hand, the example thereof shows that the information processing devices 10D to 10H are not registered as the contacts in the information processing device 10A. In the example of FIG. 10, the control unit 36 controls to transmit the user information to the information processing devices 10B and 10C in a case where the preset condition is satisfied.

In a case where the position information included in the user information indicates a residential location of the user A, the control unit 36 preferably processes the position information into position information indicating a non-residential location near the residential location of the user A. The “non-residential location” is, for example, a position where people do not live, such as a road, a river, or an agricultural land. Alternatively, in a case where the position information is represented by latitude and longitude, the control unit 36 preferably processes the position information into position information indicating a wider range than the residential location by rounding digits of latitude and longitude or the like. This is because the position information of the user A is considered to be personal information, and from the viewpoint of protecting the personal information, it is preferable to prevent the residential location of the user A from being known.

The control unit 36 may transmit a command regarding the measurement of the biological information to the user which is a transmission destination, together with the user information. For example, the control unit 36 may transmit a command to start the measurement of the biological information to the measurement device 4 that measures the biological information of the user which is the transmission destination. For example, the control unit 36 may transmit a command to change a measurement condition in the measurement of the biological information to the measurement device 4 that measures the biological information of the user which is the transmission destination. The “measurement condition” may be, for example, a type of the biological information to be measured and a period of continuing the measurement, a measurement frequency, and the like in a case where the measurement device 4 measures the biological information over time. Specifically, the “measurement condition” may be, for example, a command to change a measurement frequency setting such that the measurement device 4 which is set to measure a body temperature once a day measures the body temperature three times a day.

The health information included in the user information transmitted by the information processing device 10A is not limited to information related to the user A, which is the transmission source. As described in the first embodiment, for the second user whose position information indicates that the user has been present at the same position in the same time zone as the first user subjected to the risk estimation, the estimation unit 34 can estimate that the second user has the same risk as the first user. Thus, in a case where the user A is estimated to have the same risk as the risk estimated for another person (first user) around the user A (second user), the control unit 36 may control to transmit the user information including the risk estimated for the other person. In this case, the transmitted user information may include information indicating the health condition of the other person (for example, biological information). That is, the “health information” included in the user information may be information indicating at least one of the health condition or the risk that may affect the health or safety for at least one of the user A or another person around the user A.

In this case, in a case where the health information is acquired from another person around the user A, the control unit 36 may determine that the preset condition is satisfied control to transmit the user information to the pre-registered contact.

Next, an example of functional configurations of the information processing devices 10B to 10H on a reception side of the user information will be described. As shown in FIG. 3, similarly to the information processing device 10A and the information processing device 10 according to the first embodiment, the information processing devices 10B to 10H include the acquisition unit 30, the determination unit 32, the estimation unit 34, and the control unit 36. The CPU 21 executes the information processing program 27 to function the CPU 21 as the acquisition unit 30, the determination unit 32, the estimation unit 34, and the control unit 36.

First, as a representative, the information processing device 10B owned by the user B will be described. The acquisition unit 30 of the information processing device 10B receives the user information including the health information about the health of another person (user A) and the position information of another person (user A). The reception of the user information is performed, for example, via at least one of e-mail or SNS.

The determination unit 32 of the information processing device 10B determines whether or not the position information included in the user information acquired by the acquisition unit 30 indicates a position within a range R set in advance from at least one of a position of the own device (information processing device 10B) or a preset position. The “position of own device” is, for example, a position including a movement destination of the own device, which is positioned using GPS. The “preset position” is, for example, a position designated in advance such as a residential location, workplace, school, or the like of the user B. In the example of FIG. 10, the position information of the information processing device 10A included in the user information indicates the position within the range R set in advance from the position of the information processing device 10B.

In a case where the determination by the determination unit 32 is affirmative, the control unit 36 of the information processing device 10B transfers the user information received by the acquisition unit 30 to the pre-registered contact. In this case, the control unit 36 preferably transfers the user information to a contact that belongs to a different affiliation from the user A who is a transmission source of the user information. The transfer of the user information is performed, for example, via at least one of e-mail or SNS.

In the example of FIG. 10, in the information processing device 10B, the information processing devices 10C, 10D, and 10G are registered in advance as the contacts. However, as described above, it is mutually known in advance that the information processing devices 10A to 10C belong to the same group. In this case, the control unit 36 transfers the user information about the user A to the information processing devices 10D and 10G and does not transfer the user information about the user A to the information processing device 10C. Accordingly, it is possible to avoid duplicately transferring the user information about the user A to the information processing device 10C.

The transferred user information is preferably provided with transfer count information indicating the number of transfers of the user information, and the control unit 36 preferably increments the number of transfers indicated by the received transfer count information and then transfers the user information to which the transfer count information is added. In a case where the number of transfers indicated by the transfer count information reaches a preset threshold value, the control unit 36 preferably does not transfer the user information. This is because the more people are involved, the weaker a relationship with the user A, which is the transmission source, and the smaller the influence of the health condition of the user A, thus the value of transferring the user information is considered to be smaller. By setting a limit on the number of transfers, it is possible to prevent the transfer of the user information from looping.

In FIG. 10, the number of transfers is indicated by the circled number. As shown in FIG. 10, the control unit 36 increments, to “2”, the number of transfers indicated by the transfer count information, which is “1” at a stage of receiving the user information from the information processing device 10A, and then transfers the user information to which the information is added to the information processing devices 10D and 10G. Similarly, the information processing device 10D that has received the user information from the information processing device 10B increments, to “3”, the number of transfers indicated by the transfer count information and then transfers the user information about the user A to which the transfer count information is added to the information processing device 10F registered in advance as the contact. The threshold value regarding the number of transfers is assumed to be set to “3” times in advance. In this case, the information processing device 10F that has received the user information from the information processing device 10D does not transfer the user information since in a case where the number of transfers is further incremented, the number of transfers becomes 4 and exceeds the threshold value.

In a case where the same user information as the received user information has been received in the past, the control unit 36 preferably discards the received user information. In FIG. 10, the same processing as the information processing device 10B is performed on the information processing device 10C that has received the user information from the information processing device 10A, and the user information about user A provided with the transfer count information in which the number of transfers is incremented to “2” is transferred to the information processing device 10E. Further, the same processing is performed on the information processing device 10E that has received the user information from the information processing device 10C, and the user information about user A provided with the transfer count information in which the number of transfers is incremented to “3” is transferred to the information processing device 10F. The information processing device 10F has already received the user information about the user A from the information processing device 10D. Therefore, the control unit 36 of the information processing device 10F discards the user information received from the information processing device 10E and does not transfer the user information.

Further, the information processing device 10G that has received the user information from the information processing device 10B is located outside the range R set in advance from a position of the information processing device 10A (refer to FIG. 10). Therefore, in the information processing device 10G, negative determination is made by the determination unit 32. In this case, the control unit 36 of the information processing device 10G discards the user information without transferring the user information about the user A.

The information processing device 10H registered in advance as the contact of the information processing devices 10F and 10G is located within the range R set in advance from the position of the information processing device 10A, but the user information about the user A is not transferred from any information processing device. However, since a large number of users are present between the user H and the user A or a relationship between the user H and the user A is only via a distant user (user G), it is considered that the effect of transferring the user information of the user A is small and it is not necessary to transfer the user information.

In a case where the command related to the measurement of the biological information is received together with the user information, the control unit 36 may also transfer the command. Further, the control unit 36 may control the measurement device 4 to measure the biological information according to the command.

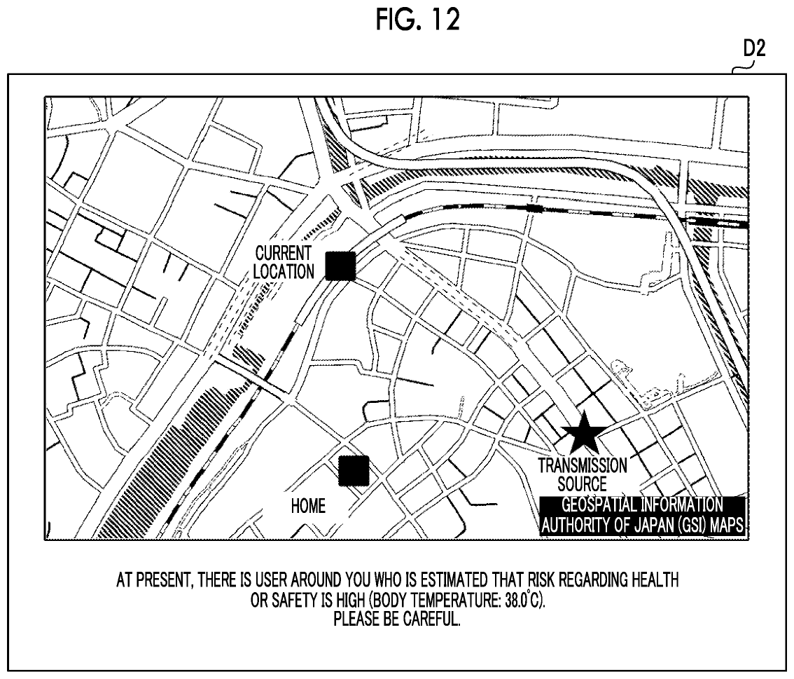

The control unit 36 may control to display the received user information of the user A on the display 24. FIG. 12 shows an example of a screen D2 displayed on the display 24 of the information processing devices 10B to 10F. The screen D2 displays the position of the user A based on the user information of the user A shown in FIG. 11 (shown by a star), the position of the own device, and a preset home position (shown by squares). The screen D2 further displays the body temperature and risk of the user A based on the user information of the user A shown in FIG. 11. By viewing the screen D2, the users B to F can check the health information of another person around the users and the position of the other person.

In a case where the control unit 36 controls to display the position of the user A (that is, the position of the transmission source of the user information) on the display 24, the control unit 36 preferably processes the position thereof such that the residential location of the user A is not known and displays the processed position thereof. For example, the control unit 36 may control to display a graphic form showing the position of the user A to be larger than the residential location and/or as an asymmetrical graphic form whose center position is difficult to understand. For example, the control unit 36 may control to display the position of the user A by a sentence indicating the non-residential location near the residential location of the user A (for example, “near X town intersection” or the like). This is because the position information of the user A is considered to be personal information, and from the viewpoint of protecting the personal information, it is preferable to prevent the residential location of the user A from being known.

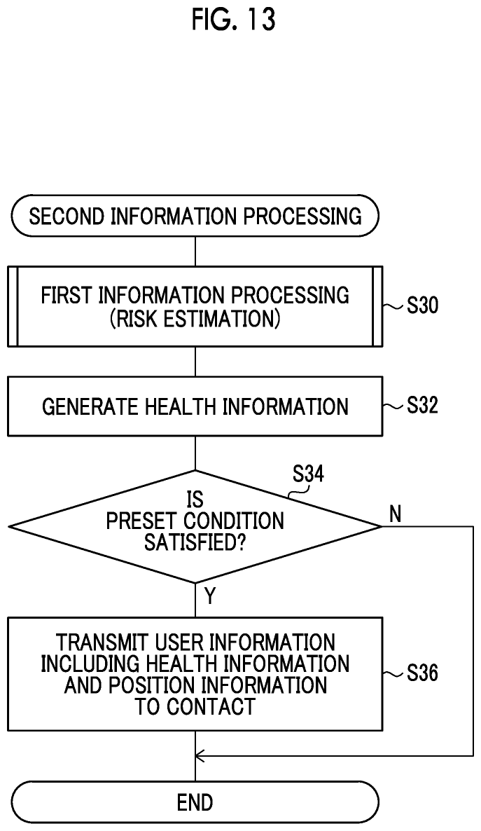

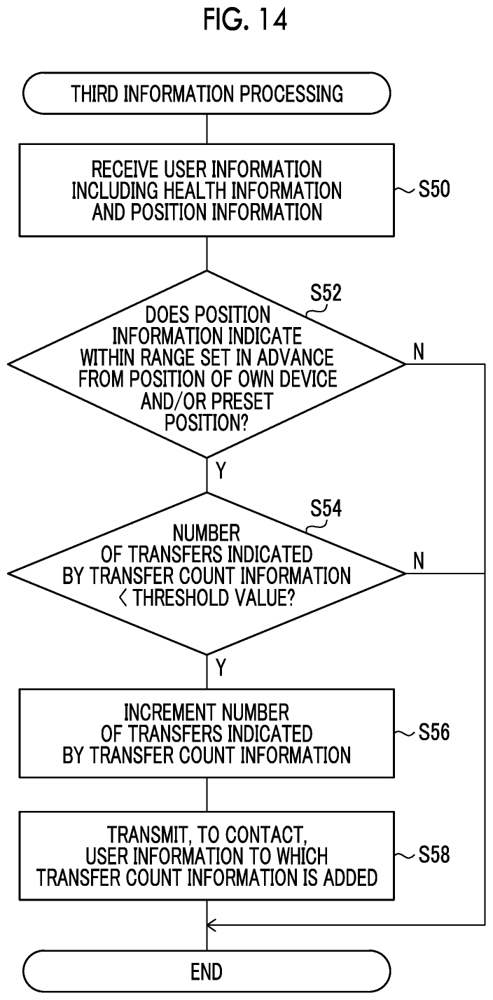

Next, an action of the information processing device 10 according to the present embodiment will be described with reference to FIGS. 13 and 14. In the information processing device 10, the CPU 21 executes the information processing program 27 to execute the second information processing shown in FIG. 13 and/or the third information processing shown in FIG. 14. The second information processing is processing in the information processing device, which is the transmission source of user information, and processing performed in the information processing device 10A in the example of FIG. 10. The second information processing is executed, for example, in a case where the user gives the instruction to start execution via the input unit 25.

In step S30, the CPU 21 executes the first information processing to estimate the risk that may affect the health or safety of the user. In step S32, the estimation unit 34 generates the health information about the user's health, including at least one of the biological information of the user acquired in step S18 or the risk estimated in step S22 in the first information processing.

In step S34, the control unit 36 determines whether or not the above preset condition is satisfied. In a case where affirmative determination is made in step S34 (that is, in a case where the preset condition is satisfied), the processing proceeds to step S36. In step S36, the control unit 36 adds the transfer count information in which the number of transfers is set to “1” to the user information including the health information generated in step S32 and the position information of the user acquired in step S14 in the first information processing and transmits the added user information to the pre-registered contact. In a case where step S36 is completed, the CPU 21 ends the second information processing. In a case where negative determination is made in step S34 (that is, in a case where the preset condition is not satisfied), the CPU 21 does not perform the processing of step S36 and ends the second information processing.

The third information processing is processing in the information processing device that has received the user information and processing performed in the information processing devices 10B to 10G in the example of FIG. 10. For example, in a case where the acquisition unit 30 receives the user information (corresponding to step S50), the third information processing is started.

In step S50, the acquisition unit 30 receives the user information including the health information of another person and the position information of another person. It is assumed that the user information is provided with the transfer count information indicating the number of transfers of the user information. In step S52, the determination unit 32 determines whether or not the position information of another person included in the user information acquired in step S50 indicates the position within the range set in advance from at least one of the position of the own device or a preset position (for example, residential location, workplace, school, or the like).

In a case where affirmative determination is made in step S52 (that is, the position information indicates the position within the range set in advance from the position of the own device and/or the preset position), the processing proceeds to step S54. In step S54, the control unit 36 determines whether or not the number of transfers indicated by the transfer count information added to the user information received in step S50 is less than the preset threshold value. In a case where affirmative determination is made in step S54 (that is, the number of transfers is less than the threshold value), the processing proceeds to step S56.

In step S56, the control unit 36 increments the number of transfers indicated by the transfer count information added to the user information received in step S50. In step S58, the control unit 36 transfers, to the pre-registered contact, the user information that is received in step S50 and in which the number of transfers is incremented in step S56 for the added the transfer count information and ends the third information processing.

On the other hand, in a case where negative determination is made in step S52 (that is, in a case where the position information indicates outside the range set in advance from the position of the own device and/or the preset position), the processing of steps S54 to S58 is not performed, and the third information processing is ended. In a case where negative determination is made in step S54 (that is, in a case where the number of transfers reaches the threshold value), the processing of steps S56 to S58 is not performed, and the third information processing is ended.

As described above, the information processing device 10 according to one aspect of the present disclosure comprises at least one processor. The processor transmits the user information including the health information about the user's health and the position information of the user to the pre-registered contact in a case where the preset condition is satisfied. That is, the user's health information can be easily shared with the pre-registered contact. Accordingly, for example, the information about the infection state of various infectious diseases such as the coronavirus disease can be quickly shared, and thus it is possible to contribute to the prevention of the spread of the infection.

The information processing device 10 according to one aspect of the present disclosure comprises at least one processor. The processor receives the user information including the health information about the health of another person and the position information of another person, determines whether or not the position information indicates the position within the range set in advance from at least one of the position of the own device or the preset position, and transfers the user information to the pre-registered contact in a case where the determination is affirmative. Therefore, even the health information of another person can be easily shared. Accordingly, for example, the information about the infection state of various infectious diseases such as the coronavirus disease can be quickly shared, and thus it is possible to contribute to the prevention of the spread of the infection.

In the above embodiment, for example, the following various processors can be used as hardware structures of processing units that execute various pieces of processing, such as the acquisition unit 30, the determination unit 32, the estimation unit 34, and the control unit 36. The various processors include a programmable logic device (PLD), such as a field programmable gate array (FPGA), which is a processor whose circuit configuration is changeable after manufacturing, a dedicated electric circuit, such as an application specific integrated circuit (ASIC), which is a processor having a circuit configuration exclusively designed to execute specific processing, and the like, in addition to the CPU which is a general-purpose processor that executes software (program) to function as various processing units, as described above.

One processing unit may be configured by one of the various processors or a combination of two or more processors of the same type or different types (for example, a combination of a plurality of FPGAs or a combination of a CPU and an FPGA). The plurality of processing units may be configured of one processor.

As an example of configuring the plurality of processing units with one processor, first, there is a form in which one processor is configured by a combination of one or more CPUs and software and the processor functions as the plurality of processing units, as represented by computers such as a client and a server. Second, there is a form in which a processor that realizes the functions of the entire system including the plurality of processing units with one integrated circuit (IC) chip is used, as represented by a system-on-chip (SoC) or the like. As described above, the various processing units are configured using one or more of the various processors as the hardware structure.

Further, more specifically, a circuitry combining circuit elements such as semiconductor elements can be used as the hardware structure of the various processors.

In the above embodiment, the form in which the information processing program 27 is stored (installed) in the storage unit 22 in advance has been described, but the present disclosure is not limited thereto. The information processing program 27 may be provided in a form of being recorded on a recording medium such as a compact disc read only memory (CD-ROM), a digital versatile disc read only memory (DVD-ROM), and a Universal Serial Bus (USB) memory. The information processing program 27 may be downloaded from an external device through a network. Further, the technique of the present disclosure extends to a storage medium that stores the information processing program non-transitorily, in addition to the information processing program.

In the technique of the present disclosure, the above embodiments can be combined as appropriate. The contents described and the contents shown hereinabove are specific descriptions regarding the part according to the technique of the present disclosure and are merely an example of the technique of the present disclosure. For example, the descriptions regarding the configurations, the functions, the actions, and the effects are descriptions regarding an example of the configurations, the functions, the actions, and the effects of the part according to the technique of the present disclosure. Accordingly, in the contents described and the contents shown hereinabove, it is needless to say that removal of an unnecessary part, or addition or replacement of a new element may be employed within a range not departing from the gist of the technique of the present disclosure.

What is claimed is:

1. An information processing device comprising at least one processor, wherein the at least one processor is configured to transmit, in a case where a preset condition is satisfied, user information including health information about health of a user and position information of the user to a pre-registered contact.

2. The information processing device according to claim 1, wherein the health information indicates at least one of a health condition or a risk that may affect health or safety for at least one of the user or another person around the user.

3. The information processing device according to claim 1, wherein the at least one processor is configured to estimate the health information based on text information input by at least one of the user or another person around the user via an input unit.

4. The information processing device according to claim 3, wherein the at least one processor is configured to estimate the health information based on a position where the user has been present within a preset period including a point in time at which the text information is input.

5. The information processing device according to claim 3, wherein the at least one processor is configured to determine that the preset condition is satisfied in at least one of a case where biological information of the user is abnormal or a case where a result of estimating the health information indicates that there is a risk that may affect health or safety of the user.

6. The information processing device according to claim 3, wherein the at least one processor is configured to determine that the preset condition is satisfied in a case where the health information is acquired from another person.

7. The information processing device according to claim 1, wherein the at least one processor is configured to transmit a command regarding measurement of biological information to a transmission destination together with the user information.

8. The information processing device according to claim 7, wherein the command is at least one of a command to start the measurement or a command to change a measurement condition in the measurement.

9. The information processing device according to claim 1, wherein the position information indicates a non-residential location near a residential location of the user.

10. An information processing device comprising at least one processor, wherein the at least one processor is configured to:
receive user information including health information about health of another person and position information of the other person;
determine whether or not the position information indicates a position within a range set in advance from at least one of a position of an own device or a preset position; and
transfer the user information to a pre-registered contact in a case where the determination is affirmative.

11. The information processing device according to claim 10, wherein the at least one processor is configured to transfer the user information to the contact that belongs to a different affiliation from a user who is a transmission source of the user information.

12. The information processing device according to claim 10, wherein the at least one processor is configured to discard the received user information in a case where the same user information as the received user information has been received in the past.

13. The information processing device according to claim 10, wherein:
transfer count information indicating the number of transfers of the user information is added to the user information, and
the at least one processor is configured to:
increment the number of transfers indicated by the received transfer count information; and then
transfer the user information to which the transfer count information is added.

14. The information processing device according to claim 13, wherein the at least one processor is configured to do not transfer the user information in a case where the number of transfers indicated by the transfer count information reaches a preset threshold value.

15. An information processing system comprising:
a first information processing device comprising at least one first processor; and
a second information processing device comprising at least one second processor, wherein:
the at least one first processor is configured to transmit, in a case where a preset condition is satisfied, user information including health information about health of a user and position information of the user to a pre-registered contact,
the at least one second processor is configured to:
receive user information including health information about health of another person and position information of the other person;
determine whether or not the position information indicates a position within a range set in advance from at least one of a position of an own device or a preset position; and
transfer the user information to a pre-registered contact in a case where the determination is affirmative.

16. The information processing system according to claim 15, wherein the transmission, reception, and transfer of the user information are performed via at least one of e-mail or social networking service (SNS).

17. An information processing method executed by a computer, the method comprising:
transmitting, in a case where a preset condition is satisfied, user information including health information about health of a user and position information of the user to a pre-registered contact.

18. An information processing method executed by a computer, the method comprising:
receiving user information including health information about health of another person and position information of the other person;
determining whether or not the position information indicates a position within a range set in advance from at least one of a position of an own device or a preset position; and
transferring the user information to a pre-registered contact in a case where the determination is affirmative.

19. A non-transitory computer-readable storage medium storing an information processing program causing a computer to execute a process comprising:
transmitting, in a case where a preset condition is satisfied, user information including health information about health of a user and position information of the user to a pre-registered contact.

20. A non-transitory computer-readable storage medium storing an information processing program causing a computer to execute a process comprising:
receiving user information including health information about health of another person and position information of the other person;
determining whether or not the position information indicates a position within a range set in advance from at least one of a position of an own device or a preset position; and
transferring the user information to a pre-registered contact in a case where the determination is affirmative.
